(19) United States
(12) Patent Application Publication
(10) Pub. No.: US 2007/0169208 A1
(43) Pub. Date: Jul. 19, 2007

(54) Silkworm strains for enabling production of proteins using various baculovirus

(75) Inventors: Takahiro Kusakabe, Fukuoka-shi (JP); Yutaka Banno, Fukuoka-shi (JP); Jae Man Lee, Fukuoka-shi (JP)

Correspondence Address: BIRCH STEWART KOLASCH & BIRCH, PO BOX 747, FALLS CHURCH, VA, 22040-0747, US

(73) Assignee: KYUSHU UNIVERSITY, NATIONAL UNVERSITY CORPORATION, Fukuoka-shi (JP)

(21) Appl. No.: 11/636,563

(22) Filed: Dec. 11, 2006

(30) Foreign Application Priority Data
Dec. 9, 2005 (JP) 2005-355871

(51) Int. Cl. A01K 67/033; C12Q 1/70; C12P 21/06; C12N 5/06

(52) U.S. Cl. 800/13; 435/5; 435/69.1; 435/348

(57) ABSTRACT

The present invention provides a method for producing a recombinant protein, characterized by using an AcNPV-susceptible silkworm and a baculovirus. The silkworm used in the present invention is preferably a silkworm of any one of the following strains i) to iv): i) a silkworm of AcNPV-susceptible strain c11 or a mutant thereof having the same biological properties as the c11 strain; ii) a silkworm of AcNPV-susceptible strain d17 or a mutant thereof having the same biological properties as the d17 strain; iii) a silkworm of AcNPV-susceptible strain f10 or a mutant thereof having the same biological properties as the f10 strain; and iv) a silkworm of AcNPV-susceptible strain f38 or a mutant thereof having the same biological properties as the f38 strain.

### BACKGROUND OF THE INVENTION

1. Technical Field

The present invention relates to silkworm strains suitable for recombinant protein production. The silkworm strains provided by the present invention are susceptible to AcNPV. The present invention is useful in the fields of mass production of recombinant proteins and functional analysis of proteins.

2. Prior Art

Production of recombinant proteins using insects has a merit of giving proteins that are modified in a manner close to mammalian proteins, and various related techniques have been developed.

In comparison with the case of using insect cells, production systems using insect individuals are advantageous in terms of reduced risk of microbial contamination and easy control of production conditions. Above all, silkworms are completely domesticated insects that are not naturally occurring, and there is no need to use a closed rearing container at each developmental stage because neither larvae nor adults escape from their rearing containers. Moreover, silkworms are easy to rear on a large scale and have a large body size, so that they are an appropriate host for recombinant protein expression.

Vectors previously used for introducing a recombinant gene into an insect system include nucleopolyhedroviruses (NPV) belonging to the Baculoviridae family, particularly Bombyx mori NPV (BmNPV) which targets silkworms as its host and is strongly pathogenic to silkworms, as well as Autographa californica NPV (AcNPV) which does not target silkworms as its host. The former has been applied not only to systems using cultured cells, but also to systems using silkworm larval individuals.

For the latter, the following techniques are known. Non-patent Document 1 reports that the use of AcNPV achieved improved efficiency in recombinant protein production under control of the polyhedrin promoter. This technique differs from conventional ones in that the untranslated region of polyhedron is integrated, and the factor combination described in this document is used in commercially available systems. Non-patent Document 2 is about the actual production of a foreign protein using the above system, and is often cited as a practical application of protein production in AcNPV-insect cell host systems. Non-patent Document 3 reports that modified AcNPV (whose host dependency is changed because it has a helicase gene from BmNPV) and silkworm larval individuals were used to produce a recombinant protein.

Non-patent Document 1: Luckow V A, Summers M D. (1988) Signals important for high-level expression of foreign genes in Autographa californica nucleopolyhedrovirus expression vectors. Virology 167, 56-71

Non-patent Document 2: V. A. Luckow, Cloning and expression of heterologous genes in insect cells with baculovirus vectors In: A. Prokop, R. K. Bajpai and C. S. Ho, Editors, Recombinant DNA Technology and Applications, McGraw-Hill, New York (1991), pp. 97-152

Non-patent Document 3: Choudary P V, Kamita S G, Maeda S. (1995) Expression of foreign genes in Bombyx mori larvae using baculovirus vectors. Methods Mol Biol. 39, 243-264

### SUMMARY OF THE INVENTION

In general, since AcNVP infects silkworms, but cannot replicate in silkworms, efficient protein expression in AcNVP-silkworm systems has been considered impossible. However, as a result of a large-scale screening for AcNVP susceptibility performed on a group of silkworm strains in which AcNVP has been considered impossible to grow, the inventors of the present invention have found multiple silkworm strains showing susceptibility to AcNVP. Also, the inventors have found that these AcNVP-susceptible silkworm strains showed a high production capacity not only in the case of using AcNVP, but also in protein production using BmNPV, thereby completing the present invention.

Namely, the present invention provides a method for producing a recombinant protein, characterized by using an AcNPV-susceptible silkworm and a baculovirus. The AcNPV-susceptible silkworm used in the production method of the present invention can be selected by a screening method comprising the following steps:

1) preparing a candidate silkworm;

2) infecting the silkworm with AcNPV to evaluate its susceptibility to ACNPV; and

3) selecting the silkworm if it is susceptible to AcNPV.

The present invention also provides such a screening method.

The term “silkworm” as used herein is intended to mean an individual of Bombyx mori unless otherwise specified, and includes not only a larval individual, but also an individual at the stage of egg, pupa, cocoon or adult.

The term “strain” as used herein for silkworms refers to a single silkworm population which can be distinguished from populations of other strains by all or a part of its properties (phenotype or genotype at the stage of egg, larva, pupa, cocoon or adult) and its origin, and which can be bred while maintaining all of its properties. The strain classification used herein follows the method proposed by the Division of Silkworm Genetics, Institute of Genetic Resources (Kyushu University, Japan). At first, strains are classified alphabetically by their primary trait and classified in more detail by using double figures (or triple figures for branch strains of the same origin).

As candidate silkworms, various silkworms such as those of various existing strains, those of hybrid strains, mutants and transformants thereof, can be provided for the screening method of the present invention. Examples include strains falling within classifications a (lethal: embryo & larvae), b (cocoon: shape & quality), c (cocoon: color), d (egg: shape & chorion), e (egg: serosa color), f (larvae: appendage & marking), g (larvae: marking), i (larvae: eye-, head-, & tail spot), k (larvae: body color), 1 (larvae: body color), m (mosaic & malformation), n (larvae: body shape), o (larvae: translucent skin), r (chromosomal aberration & % crossing), t (growth, voltinism, moltinism), u (pupa & adult), w (multi marker line) and x (new mutant in analysis). For use in recombinant protein production, preferred are strains that are relatively easy to rear. Moreover, silkworms of strains with a large body size and a high protein production capacity may be provided for this purpose.

The term “susceptibility” or “susceptible” as used herein for viruses is intended to mean the ability to be infected with the viruses and to allow the viruses to grow, unless otherwise specified. Silkworms used in the present invention are susceptible to AcNPV, and whether or not a candidate silkworm is susceptible to AcNPV may be determined, for example, by preparing a recombinant virus into which a gene for a protein allowing easy evaluation of AcNPV growth is integrated (e.g., luciferase recombinant AcNPV, GFP recombinant ACNPV), infecting a target silkworm with this virus, and evaluating the protein level in the silkworm at an appropriate timing. Silkworms of the kxs strain (Kinshu-Showa) are not infected with AcNPV or do not allow AcNPV growth, so that they are regarded as resistant to AcNPV. On the other hand, silkworms of other strains such as c11, d17, f10 and f38 described later are regarded as susceptible to AcNPV because in vivo growth of AcNPV is observed in these strains (see the Example section below). A higher degree of virus growth is more preferred for protein production; and hence, a preferred embodiment of the screening method of the present invention is to select silkworms allowing frequent growth of ACNPV.

The screening method of the present invention is suitable not only for selection of a specific silkworm individual, but also for selection of a silkworm strain.

The present invention also provides AcNPV-susceptible silkworm strains selected by the screening method of the present invention. Strains that are provided for the first time as AcNPV-susceptible ones by the present invention include a49, c11, c51, c60, d17, e15, f10, f38, g05, g30, g32, 131, 1311, 1312, n41, r02, r21, t70, w601 and fylu. The present invention also provides these strains.

Silkworm strains c11, d17, f10 and f38, which are provided by the present invention and particularly suitable for the production method of the present invention, have the following biological properties, in addition to being susceptible to AcNPV.

TABLE 1
Phenotypic classification and genetic background of strains

| Strain | c11 | d17 | f10 | f38 |
|---|---|---|---|---|
| Phenotypic | Cocoon: color | Egg: shape & | Larvae: appendage | Larvae: appendage |
| classification | | chorion | & marking | & marking |

Crossing

| | | | | | |
|---|---|---|---|---|---|
| Inherited | Egg | tsg | Se(+), Fc, Pes^{S} | Gre, Pes^{S} | |
| trait | Larva | p³Y, ns type, Amy- | p³, Amy-d^{IV}, Amy- | p³, E^{Tc}(+), Amy-d^{IV}, | P³, E^{M} (+) |
| | | d^{IV}, Amy-hcⁿ, Bph^{A}, | hc^{S}, Ict-A^{F}, Ict-D^{F}, | Amy-hc^{S}, Ict-A^{F}, Ict- | |
| | | Ict-A^{F}, Ict-D^{F}, Ict- | Ict-E^{S}, Ict-Hⁿ, Pff^{F} | D^{M}, Ict-E^{S}, Ict-Hⁿ, | |
| | | E^{F}, Ict-Hⁿ, Lp^{mn}, | | Lp-m^{S}, Pff^{F} | |
| | | Pff^{F} | | | |
| | Cocoon | C, Src(A^{M} · D^{M}), Src- | Light green, Yf, | | |
| | | 2^{M} | Src(A^{M}), Src-2^{M} | | |
| | Pupa | | | | |
| | Adult | | | | |

| | | | | |
|---|---|---|---|---|
| Remarks | Yellow cocoon | From Shinshu | H · Kp, X-ray- | (Gunma |
| | naturally occurring | University, 1950 | induced | Sericultural |
| | in Manchuria | (origin; Guangzhou | (National Institute | Experiment Station, |
| | (1925) | providence | of Genetics, 1963) | 1993) |

Columns are left blank if there is nothing to be noted, and information such as the origin and source, or the year of discovery or hybridization is shown in the remarks column.
Abbreviations in the table (a numeric in parentheses immediately following each abbreviation represents a position in the linkage map)
Egg:
Gre (1-46.4) (Green egg shell): Spontaneous; egg shell tinged yellowish green.
Fc (?) (Ferric chloride positive): Spontaneous; egg shell is dyed with FeCl2.
Se (15-16.9) (White-sided egg): Spontaneous; eggs laid by homozygote gray with wrinkles on the surface and sterile; eggs from heterozygote gray band on periphery of egg shell.
tsg (?) (temperature sensitive gray): Spontaneous; mix mottled gray eggs when kept under low or high temperature during mid-pupal stage: See also mgr.
Larva:
E (6-21.1) (Plain extra-legs): Spontaneous; extra abdominal legs on fourth and fifth segments: Thus far 35 genes known in this region comprising a large pseudoallelic series, all concerned not only with repetition or disappearance of markings and appendages but also with abnormalities of gonads, reproductive organs and nervous systems; most of them lethal as embryos in homozygotes;
Bmabd-A is deleted in E^{Ca}; BmUbx and Bmabd-A are deleted in E^{N}; E^{M} and Nc are deletion of BmAntp: Some are accompanied with chromosomal attachment reducing the number of chromosomes by one having a large set, 27 pairs in meiotic metaphase;
T(6; 7)E^{Ds}, T(6; 14)E^{Nc}, T(6; 20)E^{Np}.
p (2-0.0) (plain): Spontaneous; grown larvae white all over the body except for linear traces of crescents and star-spots: +^{P} (normal pattern): p^{B} (Black): p^{M} (Moricaud): p^{S} (Striped): p^{Sa} (Sable, X-ray induced), etc.
Cocoon:
C (12-7.2) (Outer-layer yellow cocoon): Spontaneous; in combination with Y(2-25.6), cocoon golden yellow on the outside, inside white: C^{D} (Dilute-yellow cocoon) all layers of cocoon dilute yellow: C^{I} (Inner-layer yellow cocoon) inner layer deep yellow while outer layer faintly pigmented:
C^{St} (Straw-colored cocoon) outer layer yellow when larvae homozygous, in heterozygotes pigmented faint yellow from outer to inner layer throughout: +^{C} yellow blooded white cocoon spinner.
Yf (?) (Yellow fluorescent): Spontaneous; under UV light cocoon shell shows a yellow fluorescence.
Protein, enzyme and hormone:
Amy-d (8-2.8) (Digestive juice amylase): Spontaneous; presence of four and five highly active amylase isozymes migrating toward cathode on acrylamide gel electrophoresis are respectively controlled by codominant alleles of Amy-d^{IV} and Amy-d^{V}; homozygotes for Amy-dⁿ, previously referred to as ae for weak activity, lack cathode-migrating isozymes: Weaker anode-migrating isozymes are controlled by Amy-da alleles.
Amy-hc (19-4.2) (Hemolymph amylase C): Spontaneous; hemolymph amylase can be separated into A to F bands on gel electrophoresis migrating toward anode; major band C controlled by Amy-hc alleles involving variations of migration rate as -hc^{S}(Slow), -hc^{M}(Moderate), -hc^{F}(Fast) and of activity as -hc^{W}(Weak); homozygotes for Amy-hcⁿ(null), formerly referred to as be for low activity, lack C isozyme band.
Bph (23-0.0) (Blood acid phosphatase): Spontaneous; acid phosphatase isozymes of larval hemolymph; isozymes A, B, C, D, E in the order of faster mobility are controlled by Bph^{A}˜Bph^{E} alleles, respectively; isozyme band lacking in Bph^{O}.
Ict-A (2-23.7) (Inhibitorofchymotrypsin A): Spontaneous; chymotrypsin-inhibitor protein of hemolympli, complex alleles of at least two subfractions on electrophoresis, most fastly moving region A^{F} and A^{S}, and most slowly migrating region H^{F}, H^{M}, H^{S} and null Hⁿ alleles.
Ict-D (2-23.7) (Inhibitorofchymotrypsin D): Spontaneous; chymotrypsin-inhibitory protein of hemolymph migrating moderately on gel electrophoresis; most slowly migrating band by Ict-D^{R}, slow by Ict-D^{S}, intermediate by Ict-D^{M}, fast by Ict-D^{F} allele, respectively.
Ict-E (22-0.0) (Lnhibitorofchymotrypsin E): Spontaneous; chymotrypsin-inhibitory protein of hemolymph migrating between moderately moving D-group (19-29.1) and most slow group of H (2-23.7), faster moving band by -E^{F} and slower by -E^{S} alleles.
Lp (20-6.2) (Lipoprotein): Spontaneous; low-molecular weight lipoprotein fractions of hemolymph, 30K protein, complex alleles of three subfractions separable on gel electrophoresis, fastly moving f-region with variations of A, B, C, moderately moving m-region with null type, slowly migrating s-region with variations of A, B, and duplicated forms of AB, BC.
Pes (19-0.0) (Egg specific protein): Spontaneous; one of the major components of yolk protein synthesized in ovary as against others have hemolymph origin; pes^{F} for rapidly and Pes^{S} for slowly migrating band on gel electrophoresis. See also vit.
Pfl (23-8.6) (Larval female protein): Spontaneous; female-specific protein of larval hemolymph, FL-1, having storage role at larval-pupal metamorphosis, hence also designated as SP-1; fast, moderate and slow mobilities on gel electrophoresis are controlled by Pfl^{F}, Pfl^{M} and Pfl^{S}, respectively.
Src(11-2.2) (Sericin complex): Spontaneous; cluster of genes controlling three subfractions of sericin; A migrates slowly in parallels with B, and D fastly on gel electrophoresis; F (fast), M (moderate) and S (slow) types in each subfraction; B and D trace in floss.
Src-2(11-11.4) (Sericin 2): Spontaneous; complex alleles controlling sericin-2 (C) subtraction migrating moderately with variations V (very fast), F (fast), M (moderate) and S (slow); and E-subfraction rich in the thread spun during feeding stage and before entering into molt but trace in cocoon thread with variations F and M.
Reference: Hiroshi Fujii ed., GENETICAL STOCKS AND MUTATIONS OF BOMBYX MORI: IMPORTANT GENETIC RESOURCES, Second Edition, published by the Division of Silkworm Genetics, Institute of Genetic Resources, School of Agriculture, Kyushu University (1998)

The scope of the present invention includes silkworm strains c11, d17, f10 and f38, as well as mutants thereof having the same biological properties as the c11, d17, f10 or f38 strain.

Rearing and breeding of these strains may be carried out under conditions commonly used by those skilled in the art.

The silkworm strains provided by the present invention are available for distribution from the Institute of Genetic Resources Center, Kyushu University (Faculty of Agriculture, Graduate School of Kyushu University; 6-10-1 Hakozaki, Higashi-ku, Fukuoka, 812-8581 Japan; (Tel) 092-621-4991; (Fax) 092-624-1011), which is a central institution for the National BioResource Project (NBRP), in accordance with Rule 27(3) of the regulations under the Japanese Patent Law or in accordance with Rule 11 of the regulations under the Budapest Treaty (see http://www.nbrp.jp/report/reportProject.jsp;jsessionid=BE73451C6E54680014762FD194C0F721?project=silkworm).

The AcNPV-susceptible silkworms obtained by the screening method of the present invention may also have high susceptibility to BmNPV. For this reason, they may also be useful in recombinant protein production using other baculovirus-insect systems in addition to AcNPV-based systems. Baculoviruses used in the method of the present invention for recombinant protein production are preferably nucleopolyhedroviruses, and more preferably BmNPV or AcNPV and mutants thereof.

The production method of the present invention generally comprises the steps of: preparing a recombinant virus; infecting a silkworm with the virus; growing the virus in the silkworm body to produce a desired protein; and collecting the desired protein from the silkworm.

The step of preparing a recombinant virus may be accomplished by using conventional techniques. Since the genome of baculoviruses is usually in the form of 130 kbp cyclic DNA, a target gene cannot be introduced directly. For this reason, a target gene should be pre-integrated into a transfer plasmid and then introduced into the viral genome by homologous recombination. In this regard, various techniques have been developed. In the present invention, a commercially available construction system may be used.

Although a recombinant virus may usually be prepared by integration of a foreign gene into BmNPV or AcNPV, not only BmNPV or AcNPV, but also mutants thereof such as those modified to lack a specific gene for inhibition of disease development or those modified to overexpress a specific gene for facilitation of cell entry can be used in the present invention.

A protein gene to be integrated into the viral genome can be selected from various types of proteins such as enzymes, kinases, proteases, cytokines, hormones, receptors, channels, transcription factors and virus-constituting proteins. Alternatively, it is also possible to select proteins derived from a wide variety of organisms, including mammals, viruses, insects, plants, yeast, humans, jellyfishes, corals, etc.

If necessary, the recombinant virus may be propagated in cultured cells before use in the next infection step. In addition, the resulting virus may be used in the form of a suspension, a lyophilized powder or the like, as necessary.

The recombinant virus carrying a target gene may be infected into the silkworms of the present invention at about the 5th larval instar. Conditions such as the dose of inoculation and the route of infection may be determined as appropriate by those skilled in the art. For example, 10 μl of a recombinant virus solution adjusted to 1×10⁶ pfu/ml may be injected subcutaneously into the chest using an injection needle. In terms of reducing the labor required for inoculation, it will also be useful to orally inoculate the recombinant virus in admixture with artificial diet (see Japanese Patent No. 3030430).

To rear the silkworms of the present invention before infection and to rear the infected silkworms in the step of growing the virus in the silkworm body to produce a desired protein, either artificial diet or mulberry leaves may be used.

The timing at which a desired protein is collected from the silkworms of the present invention may be determined by the concentration of the recombinant protein in the silkworm's body fluid, the absolute amount of the desired protein per individual of silkworm, etc. In general, since larvae increase their body size with the passing of days, the absolute amount of protein production per individual is increased accordingly. On the other hand, however, dead individuals may appear after the 7th day. As to the state of the silkworms of the present invention at the timing of protein collection, their appearance is substantially the same as that of uninfected silkworms, but some silkworm strains may have a slight difference in their appearance. In general, virus-infected silkworms do not become pupae even when reared for a long period of time.

A desired protein can be collected from every tissue of silkworm individuals. In the case of a secretion system, any body fluid containing a secreted protein may be collected.

For example, the collection of a desired protein may be accomplished as follows: body fluid is directly obtained from each individual using an injection needle or the like; individuals are ground in the presence of a suitable solution, as appropriate; or individuals are frozen and thawed to extract their body fluid by means of a contraction phenomenon.

The collected fluid containing a desired protein may be provided for further steps, if necessary, such as separation, purification, lyophilization, crystallization, etc.

The present invention enables the diversification of recombinant protein production using baculovirus-silkworm systems. The present invention facilitates mass production of proteins derived from various organisms, and also facilitates simultaneous production of very many kinds of proteins. For this reason, the present invention is useful for mass production of recombinant proteins and functional analysis of proteins.

The amount of a recombinant protein obtainable by the present invention will vary depending on the type of strain to be used and the type of protein to be obtained. For example, it may be 75 μg/individual for luciferase and 200 μg/individual for GFP. Such an amount is remarkably high when compared to conventional strains, each of which allows protein production at 1 μg/individual or less. Moreover, when modified into a secretion system, the production yield can be further increased. However, it should be noted that some proteins (e.g., nucleoproteins and membrane proteins) may lose their activity in the case of a secretion system. Likewise, attention should also be directed to problems of deactivation and protein insolubilization when a protein is produced in the form of silkworm cocoon. In the case of toxic proteins, their yield may be reduced.

The silkworm strains of the present invention have a uniform genetic background because pure-line crossing is repeated for each strain. For this reason, the strains of the present invention are advantageous in that there is little variation in protein production capacity among individuals within the same strain.

The silkworm strains of the present invention are useful as long as they use baculovirus-based expression systems.

### BRIEF DESCRIPTION OF THE DRAWINGS

FIG. 1 is a graph showing luciferase activity in each silkworm strain. Each silkworm strain (5th instar, day 3) was inoculated with AcNPV carrying a luciferase expression cassette, and blood cells were then collected from the body fluid taken after 3 days. The luminescence intensity of luciferase was measured for each cell extract.

FIG. 2A presents photographs showing a comparison between d17 strain (the strain of the present invention) and p50 strain (standard strain) when a fluorescent protein DsRed was expressed.

FIG. 2B presents photographs showing a comparison of each dissected tissue between d17 strain (the strain of the present invention) and p50 strain (standard strain) when a fluorescent protein DsRed was expressed.

### WORKING EXAMPLE 1

1. Material and Method

1.1 Preparation and Propagation of Recombinant Virus

A Mamestra brassicae-derived cultured cell line Sf9 and a silkworm-derived cultured cell line BmN4 were each cultured in 10% FBS-containing Grace's Insect Medium (Gibco BPL) at 23° C. before use in the test. For cell culturing, 10% FBS-containing Grace's Insect Medium was used and incubation was carried out at 23° C. All manipulations were aseptically performed in a clean bench. First, old medium was removed from flasks containing the cultured cells and replaced by fresh medium (8 ml), followed by pipetting to disperse the cells into a single cell state. Medium (7 ml) was dispensed into new flasks and the dispersed cells (1 ml) were added to give a total volume of 8 ml. The flasks were transferred to an incubator and cultured.

For construction of Bacmid DNA, a Bac-to-Bac system (Invitrogen) was used. First, pENTR11/luciferase and pFastBacA3(Gm^{r}) were prepared, between which site-specific recombination was then caused using a Gateway LR system (Invitrogen) to obtain pFastBacA3/luciferase. pENTR11/luciferase carries the full-length of firefly luciferase cDNA inserted into a NcoI-XhoI site of pENTR11 (Invitrogen), while pFastBacA3 carries a DEST cassette (Invitrogen) inserted downstream of the silkworm actin promoter. The DEST cassette can transfer a target gene on pENTR11 by Gateway LR reaction. pFastBacA3 has an attTn7-recognition sequence for transfer into Bacmid DNA and a gentamicin resistance gene as a selective marker.

The luciferase expression cassette on pFastBacA3/luciferase was introduced into the AcNPV genome using a Bac-to-Bac Baculovirus Expression System (Invitrogen). First, pFastBacA3/luciferase was transformed into DH10Bac, incubated at 37° C. for 1 hour, and then inoculated on a LB plate containing kanamycin, gentamicin, tetracycline, Bluo-gal and IPTG. After standing at 37° C. for 1 day, white colonies were collected with a platinum loop and cultured in 1.5 ml LB medium.

Subsequently, Bacmid DNA extraction was performed. The extraction of Bacmid DNA was accomplished by the alkali-SDS method. The culture solution (1.5 ml) was centrifuged at 14,000× g for 1 minute to collect the cells, which were then suspended and vortexed completely in 300 μl of 10 mM EDTA, 15 mM Tris-HCl (pH 8.0), 100 mg/ml RNase A. Next, after 300 μl of 0.2 N NaOH, 1% SDS was added and mixed gently, the mixture was allowed to stand at room temperature for 5 minutes. After 300 μl of 3 M potassium acetate (pH 5.5) was further added and mixed well, the mixture was allowed to stand in ice for 5 minutes. Centrifugation was then performed at 4° C. at 14,000 rpm for 10 minutes to collect the supernatant, followed by isopropanol precipitation. The precipitate was dissolved in 40 μl of a solution (TE) containing 1 M Tris (pH 8.0) and 0.5 M EDTA (pH 8.0), and used as a Bacmid sample.

Transfection into Sf9 and BmN4 cells was performed as follows. The Sf9 cells were used as host cells for ACNPV propagation, while the BmN4 cells were used as host cells for BmNPV propagation. On the day before transfection, the cells were dispersed and seeded in 24-well plates at 1×10⁵ cells/well, followed by culturing at 23° C. for 1 day. Bacmid DNA (0.5 μg) was diluted with HBS to 10 μl per well and used as Solution A. At the same time, 4 μl of CellFectin (GIBCO) was diluted with 16 μl HBS and incubated on ice for 45 minutes. During a waiting period of 45 minutes, the cells were washed once with SFM (GIBCO). To form a conjugate between the cation lipid and the DNA, incubation was continued in ice for an additional 15 minutes. After addition of 170 μl SFM to the mixture, the medium in each well was removed and the mixture was added to the cells. After culturing at 23° C. for 8 hours, the transfection mixture was removed and replaced by 1 ml of 10% FBS-containing Grace's Insect Medium (Gibco BPL), followed by further culturing. After culturing for 3 days at 270°, the culture solution was collected and sterilized by filtration, which was used as a baculovirus-containing solution (P1 virus solution). This P1 virus solution (10 μl) was infected into 5×10⁵ host cells and, after 3 days, the virus solution was collected (P2 virus solution). The P2 virus solution was also amplified in the same manner to give a P3 virus solution, which was then used for the inoculation experiment on silkworm larvae.

1.2 Virus Inoculation

In this experiment, 163 silkworm strains belonging to the Faculty of Agriculture, Kyushu University were used, and 10 individuals per strain received virus inoculation.

Silkworm larvae (5th instar, day 3) were inoculated via injection needles with the P3 virus solution (10 μl) adjusted to 1×10⁶ pfu/ml. The third day after inoculation was selected as a time point where the recombinant protein level in the body fluid would reach a plateau, and a needle was stuck into a leg of each larva to obtain two drops of its body fluid. The body fluid was centrifuged and only the precipitated blood cells were collected. It should be noted that the silkworm larvae were reared in a standard manner using mulberry leaves throughout the experimental period.

1.3 Virus Growth in Silkworm Larvae (Measured as Luciferase Activity)

Each body fluid sample collected was transferred to a 1.5 ml Eppendorf tube. This tube was centrifuged at 4° C. at 2,000 rpm for 1 minute to collect blood cells present in the body fluid. After,removal of the culture solution, the cells were washed once with PBS. A cell lysis solution (100 μl) was added and the cells were allowed to stand at room temperature for 15 minutes or longer. After centrifugation at 4° C. at 10,000 rpm for 30 seconds, a luminescence substrate solution (100 μl) was added to each cell extract (80 μl) to measure the luminescence intensity of luciferase using a luminometer. The remaining 20 μl cell lysate solution was subjected to protein quantification for correction of luciferase activity.

2. Results

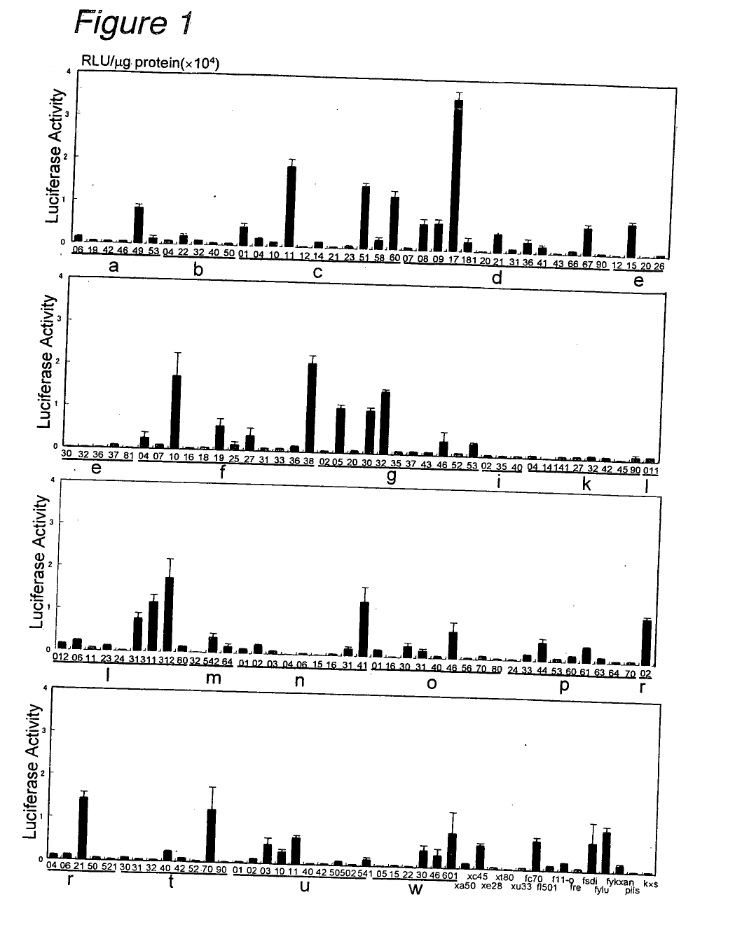

The results obtained are shown in FIG. 1. Multiple strains (e.g., a49, c11, c51, c60, d17, e15, f10 , f38, g05, g30, g32, 131, 1311, 1312, n41, r02, r21, t70, w601 and fylu strains) were susceptible to AcNPV, and particularly c11, d17, f10 and f38 strains were found to have a high protein production capacity.

The data obtained for these 4 strains during the initial screening are shown in the table below.

| Strain | Luc activity | | | | | | | | Mean |
|---|---|---|---|---|---|---|---|---|---|
| c11 | 3441 | 4198 | 2872 | 3957 | 3553 | 2713 | 3753 | | 3498 |
| d17 | 6171 | 7774 | 7166 | 6358 | 4393 | 6859 | 7688 | 5185 | 6449 |
| f10 | 1796 | 659 | 974.1 | 5571 | 1491 | 117.3 | | | 1768 |
| f38 | 6007 | 4231 | 549.5 | 4891 | 7083 | 1770 | | | 4089 |
| ks | 18.41 | 38.66 | 18.83 | 13.69 | 6.535 | 9.883 | 21.02 | | 18.15 |

Moreover, further test data obtained for each al in two susceptible strains and two resistant are shown in the table below.

| | Strain | | | | | | | | | | | |
|---|---|---|---|---|---|---|---|---|---|---|---|---|
| | d17 | f38 | ks | w05 | d17 | f38 | ks | w05 | d17 | f38 | ks | w05 |
| Luc activity | 6171 | 6007 | 119.5 | 5.463 | 5117 | 1891 | 34.08 | 28.66 | 3320 | 2455 | | 2.502 |
| | 7774 | 4231 | 179 | 8.326 | 3294 | 4324 | 53.24 | 12.96 | 2582 | 2228 | | 5.594 |
| | 7166 | 549.5 | 118 | 18.32 | 1874 | 2284 | 21.19 | 0.203 | 3946 | 3192 | | |
| | 6358 | 4891 | 112 | 12.05 | 3465 | 3378 | 44.31 | 17.42 | 2726 | 1732 | | |
| | 4393 | 7083 | 50.3 | 17.97 | 3229 | 1539 | 43.43 | 6.166 | 2086 | 634.1 | | |
| | 6859 | 1770 | 49.59 | 29.08 | 1732 | 2335 | 20.4 | 0.466 | 2257 | | | |
| | 7688 | 2240 | 40.37 | 10.75 | 5663 | 2631 | 73.66 | 55.43 | 3079 | | | |
| | 5185 | 3220 | 142.1 | 57.14 | 5435 | 2046 | 9.26 | 3.776 | 3556 | | | |
| | 2278 | 2662 | 60 | 56.76 | 3999 | 2605 | 25.71 | 3.408 | 3948 | | | |
| | 3035 | 2987 | 18.41 | 22.38 | 4639 | 2324 | 29.72 | 28.89 | 2125 | | | |
| | 3475 | 3569 | 38.66 | 61.06 | 5630 | 3149 | 45.63 | 11.82 | 2971 | | | |
| | 3861 | 234.4 | 18.83 | 34.95 | 4129 | 2060 | 29.75 | 30.22 | 3673 | | | |
| | 113.2 | 3464 | 13.69 | 43.9 | 651.8 | 2116 | 15.53 | 12.11 | 2226 | | | |
| | 3566 | 2185 | 6.535 | 17.42 | 3473 | 2577 | 61.39 | 4.882 | 3412 | | | |
| | 3355 | 3710 | 9.883 | 29.59 | 2419 | 2833 | 34.35 | 2.337 | 2542 | | | |
| | 4416 | 2179 | 21.02 | 24.31 | 2742 | 2565 | 24.05 | 11.01 | 3172 | | | |
| | 2722 | 2541 | 20.46 | 62.67 | 2688 | 2957 | 40.87 | 16.84 | 2244 | | | |
| | 3069 | 3285 | 56.33 | 17.67 | 1508 | 2363 | 21.6 | 21.2 | 1135 | | | |
| | 3677 | 3255 | 12.92 | 24.14 | 5575 | 1689 | 22.37 | 9.95 | 1450 | | | |
| | 3122 | 2426 | 5.485 | 6.618 | 2881 | 1977 | 36.12 | 13.65 | 2163 | | | |
| | 3001 | 1661 | 446.7 | 38.95 | 2768 | 3123 | 47.55 | 11.74 | 443.4 | | | |
| | 3540 | 3146 | 31.13 | 21.31 | 1829 | 973.6 | 15.51 | 5.134 | 1115 | | | |
| | 1832 | 1783 | 3.982 | 54.88 | 2207 | 3239 | 19.28 | 6.504 | 1223 | | | |
| | 3591 | 3647 | 6.567 | 23.6 | 1928 | 2103 | 29.5 | 8.294 | 1444 | | | |
| | 3881 | 5852 | 4.347 | 59.07 | 4448 | 1811 | 21.16 | 7.915 | 1012 | | | |
| | 3139 | 2551 | 41.67 | 44.04 | 3186 | 2627 | 32.62 | 3.503 | 1415 | | | |
| | 3348 | 2678 | 89.31 | 25.68 | 3238 | 2015 | 44.21 | 0.168 | 719.8 | | | |
| | 1597 | 3902 | 61.74 | 5.461 | 3769 | 1533 | | 3.113 | 1363 | | | |
| | 3833 | 2248 | 21.44 | 69.88 | 5151 | 1415 | | 8.011 | 1414 | | | |
| | 3543 | 2923 | 84.14 | 38.35 | 1940 | 1662 | | 0.998 | 1311 | | | |
| Mean | | | | | | | | | 3181 | 2666 | 48.78 | 20.91 |

It could be confirmed that there were small variations among individuals in each silkworm strain.

### WORKING EXAMPLE 2

Recombinant BmNPV was used to express a fluorescent protein DsRed in both d17 strain and p50 strain (standard strain). Test procedures were carried out as in Example 1.

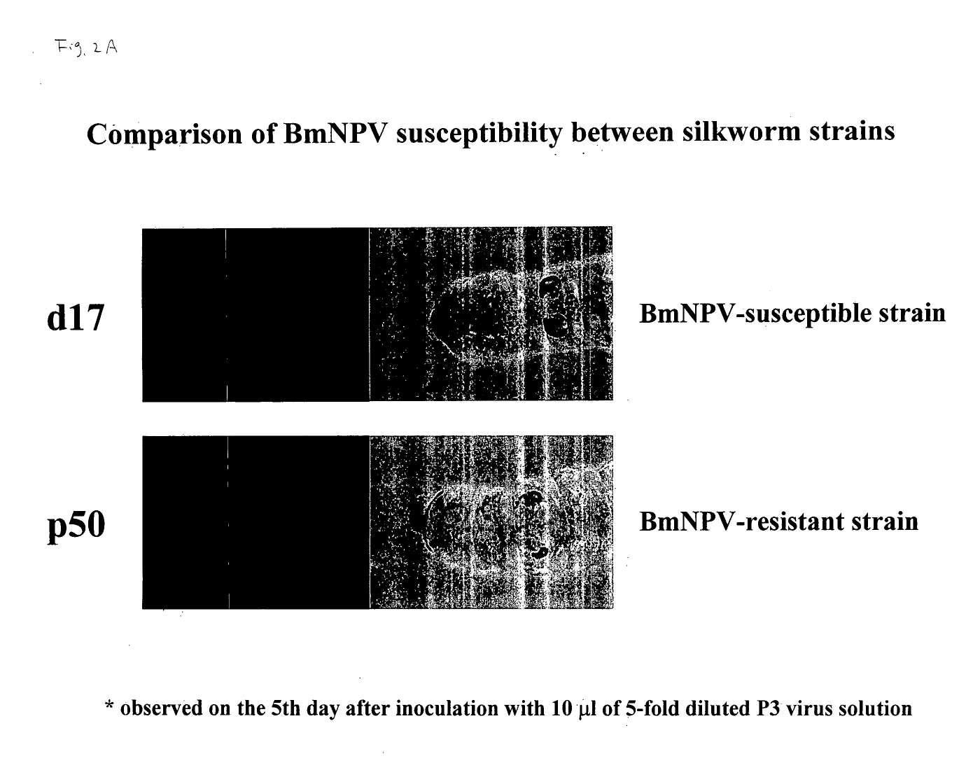

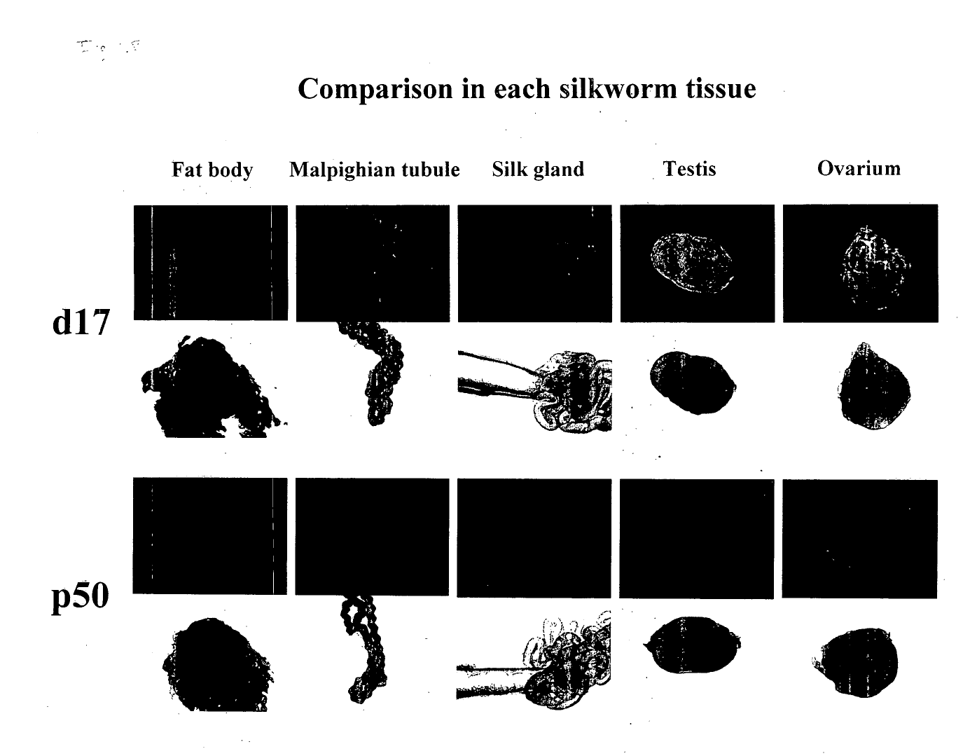

The results obtained are shown in FIGS. 2A and 2B. When a fluorescent protein DsRed was expressed, the d17 strain (the strain of the present invention) was found to have a high protein production capacity in every tissue in the body, as compared to the p50 strain (standard strain). This indicates that the silkworm strains of the present invention are useful as long as they use baculovirus-based expression systems.

1. A method for producing a recombinant protein, which uses an AcNPV-susceptible silkworm and a baculovirus.

2. The method according to claim 1, wherein the silkworm is any one of i) to iv) shown below:
i) a silkworm of AcNPV-susceptible strain c11 or a mutant thereof having the same biological properties as the c11 strain;
ii) a silkworm of AcNPV-susceptible strain d17 or a mutant thereof having the same biological properties as the d17 strain;
iii) a silkworm of AcNPV-susceptible strain f10 or a mutant thereof having the same biological properties as the f10 strain; and
iv) a silkworm of AcNPV-susceptible strain f38 or a mutant thereof having the same biological properties as the f38 strain.

3. A method for screening a silkworm for use in baculovirus-based recombinant protein production, which comprises the following steps:
1) preparing a candidate silkworm;
2) infecting the silkworm with AcNPV to evaluate its susceptibility to AcNPV; and
3) selecting the silkworm if it is susceptible to AcNPV.

4. A method for producing a recombinant protein, which uses a silkworm selected by the method according to claim 3 and a baculovirus.

5. The method according to claim 1 or 3, wherein the baculovirus is AcNPV.

6. A method for producing a silkworm for use in baculovirus-based recombinant protein production, which comprises the following steps:
1) preparing a candidate silkworm;
2) infecting the silkworm with AcNPV to evaluate its susceptibility to AcNPV;
3) selecting the silkworm if it is susceptible to AcNPV; and
4) breeding the selected silkworm.

7. A silkworm strain which is any one of i) to iv) shown below:
i) a silkworm of AcNPV-susceptible strain c11 or a mutant thereof having the same biological properties as the c11 strain;
ii) a silkworm of AcNPV-susceptible strain d17 or a mutant thereof having the same biological properties as the d17 strain;
iii) a silkworm of AcNPV-susceptible strain f10 or a mutant thereof having the same biological properties as the f10 strain; and
iv) a silkworm of AcNPV-susceptible strain f38 or a mutant thereof having the same biological properties as the f38 strain.
